(19) United States
(12) Patent Application Publication
(10) Pub. No.: US 2020/0018740 A1
(43) Pub. Date: Jan. 16, 2020

(54) METHOD, APPARATUS AND SYSTEM FOR DETERMINING SWEET SPOT REGION FOR SHALE OIL IN-SITU CONVERSION DEVELOPMENT

(71) Applicant: PetroChina Company Limited, Beijing (CN)

(72) Inventors: Lianhua Hou, Beijing City (CN); Jinhua Fu, Beijing City (CN); Xia Luo, Beijing City (CN); Xianyang Liu, Beijing City (CN); Lijun Zhang, Beijing City (CN); Shixiang Li, Beijing City (CN); Senhu Lin, Beijing City (CN); Zhi Yang, Beijing City (CN); Qian Zou, Beijing City (CN); Jingwei Cui, Beijing City (CN); Songtao Wu, Beijing City (CN); Jinghong Wang, Beijing City (CN)

(21) Appl. No.: 16/509,348

(22) Filed: Jul. 11, 2019

(30) Foreign Application Priority Data
Jul. 12, 2018 (CN) 201810763086.1

(51) Int. Cl. G01N 33/28; G06Q 40/06

(52) U.S. Cl. CPC G01N 33/2841; G06Q 40/06

(57) ABSTRACT

The embodiments of the present disclosure disclose a method, apparatus and system for determining sweet spot region for shale oil in-situ conversion development. The method comprises: determining an output oil and gas potential index according to a Total Organic Carbon (TOC), a Hydrogen Index (HI) and a shale density; determining a heated shale section according to the output oil and gas potential index and corresponding lower limit value of the oil and gas potential index that is determined according to a well arrangement mode and a shale vitrinite reflectance; determining an output quantity according to a thickness and an area of the heated shale section and data of the output oil and gas potential index; determining a Return on Investment (ROI) according to the output quantity and an invested cost; and determining a sweet spot region for shale oil in-situ conversion development by using the ROI.

### INCORPORATION BY REFERENCE

An Application Data Sheet is filed concurrently with this specification as part of the present application. Each application that the present application claims benefit of or priority to as identified in the concurrently filed Application Data Sheet is incorporated by reference herein in its entirety and for all purposes

### TECHNICAL FIELD

The present disclosure relates to the technical field of exploration and development, and in particular, to a method, an apparatus and a system for determining a sweet spot region for shale oil in-situ conversion development.

### BACKGROUND ART

The shale oil has become an important field in the global petrolatum exploration and development, but the exploration and development practices has proved that when the vitrinite reflectance (Rₒ) of the shale is less than 0.95%, a scale-benefit development cannot be achieved with the existing horizontal well volume fracturing technology. Currently, the shale oil is usually developed with an in-situ conversion technology which performs a development by converting unconverted organic matters and generated hydrocarbons in shale with low and medium maturities into lightweight oil and natural gas by using an in-situ electric heating method.

Currently, the favorable section is usually determined by using a product of a shale oil output quantity and a shale thickness, or a product of Total Organic Carbon (TOC) in shale and a shale thickness, so as to realize the evaluation and optimization of the sweet spot region for shale oil development. However, the above methods only evaluate the sweet spot region for shale oil development based on geological factors, and the evaluation results are not accurate enough. Therefore, there is an urgent need in the industry for a method capable of determining the shale oil sweet spot region more accurately.

### SUMMARY OF THE DISCLOSURE

An objective of the embodiments of the present disclosure is to provide a method, apparatus and system for determining a sweet spot region for shale oil in-situ conversion development, so as to more accurately determine the sweet spot region for the shale oil development.

The present disclosure provides a method, apparatus and system for determining a sweet spot region for shale oil in-situ conversion development as follows:

A method for determining a sweet spot region for shale oil in-situ conversion development, comprising:

determining an output oil and gas potential index according to a Total Organic Carbon (TOC), a Hydrogen Index (HI) and a shale density;

determining a heated shale section according to the output oil and gas potential index and corresponding lower limit value of the oil and gas potential index that is determined according to a well arrangement mode and a shale vitrinite reflectance;

determining an output quantity according to a thickness and an area of the heated shale section and data of the output oil and gas potential index;

determining a Return on Investment (ROI) according to the output quantity and an invested cost; and

determining a sweet spot region for shale oil in-situ conversion development by using the ROI.

In another embodiment of the method provided by the present disclosure, the determining a heated shale section according to the output oil and gas potential index and corresponding lower limit value of the oil and gas potential index comprises:

determining an interval, where the output oil and gas potential index is greater than or equal to corresponding lower limit value of the oil and gas potential index, as an effective shale section;

determining a continuous shale interval, where a ratio of a thickness of the effective shale to thicknesses of the effective shale section and an interlayer between the effective shale sections is greater than a preset threshold, as the heated shale section.

In another embodiment of the method provided by the present disclosure, the determining a heated shale section according to the output oil and gas potential index and corresponding lower limit value of the oil and gas potential index comprises:

determining the number of well arrangement layers of a shale section to be evaluated, according to the lower limit values of the oil and gas potential indexes corresponding to different numbers of well arrangement layers and upper and lower limit values of a shale thickness;

determining the lower limit value of the oil and gas potential index of the shale section to be evaluated, according to the number of well arrangement layers of the shale section to be evaluated and a shale vitrinite reflectance; and determining the heated shale section of the shale section to be evaluated, according to the output oil and gas potential index and the lower limit value of the oil and gas potential index of the shale section to be evaluated.

In another embodiment of the method provided by the present disclosure, the determining an output quantity according to a thickness and an area of the heated shale section and data of the output oil and gas potential index comprises:

determining an output rate of the heated shale section according to data of the output oil and gas potential index and ratio of the output rate of the heated shale section; and

calculating the output quantity according to the output rate of the heated shale section as well as the thickness, the area, and the shale density thereof.

In another embodiment of the method provided by the present disclosure, the well arrangement mode includes: a heating well pattern perpendicular to a stratum section is arranged by using a linear pattern in single layer or a triangular pattern in two or more layers.

In another embodiment of the method provided by the present disclosure, the determining the lower limit value of the oil and gas potential index according to the number of well arrangement layers and a shale vitrinite reflectance comprises:

determining the lower limit value of the oil and gas potential index of a target stratum according to a pre-built calculation model for the lower limit value of the oil and gas potential index, the calculation model includes:

PHI_{cutof}=100×(a₈₅×Ro⁵+a₈₄×Ro⁴+a₈₃×Ro³+a₈₂×Ro²+a₈₁×Ro+a₈₀)

wherein PHI_{cutof} represents a lower limit value of an oil and gas potential index, Ro represents a vitrinite reflectance, and a₈₀, a₈₁, a₈₂, a₈₃, a₈₄ and a₈₅ represent constants and are determined according to the number of well arrangement layers.

In another embodiment of the method provided by the present disclosure, upper and lower limit values of the shale thickness are calculated according to a shale thickness calculation model as follows:

$${H}_{up} or {H}_{down} = \{ \begin{matrix}{a}_{33} \times {NL}^{3} + {a}_{32} \times {NL}^{2} + {a}_{31} \times NL + {a}_{30} & NL \leq 4 \\ {b}_{31} \times NL + {b}_{30} & NL > 4\end{matrix}$$

wherein NL represents the number of well arrangement layers of a heating well, Hᵤₚ represents an upper limit value of a shale thickness of corresponding to NL, H_{down} represents a lower limit value of the shale thickness of the corresponding to NL, and a₃₃, a₃₂, a₃₁, a₃₀, b₃₁ and b₃₀ represent constants.

In another embodiment of the method provided by the present disclosure, a ratio of the output rate of the heated shale section is calculated according to a pre-built percentage calculation model as follows:

$$\{ \begin{matrix}PRo = 100 \times ( {a}_{46} \times {Ro}^{6} + {a}_{45} \times {Ro}^{5} + {a}_{44} \times {Ro}^{4} + {a}_{43} \times {Ro}^{3} + {a}_{42} \times {Ro}^{2} + {a}_{41} \times Ro + {a}_{40} ) \\ PRg = 100 \times ( {a}_{53} \times {Ro}^{3} + {a}_{52} \times {Ro}^{2} + {a}_{51} \times Ro + {a}_{50} )\end{matrix}$$

wherein PRO represents a ratio of oil output rate, PRg represents a ratio of gas output rate, Ro represents a shale vitrinite reflectance, and a₄₀, a₄₁, a₄₂, a₄₃, a₄₄, a₄₅, a₄₆, a₅₀, a₅₁, a₅₂ and a₅₃ represent constants.

In another embodiment of the method provided by the present disclosure, the determining a Return on Investment (ROI) according to the output quantity and an invested cost comprises:

$$\{ \begin{matrix}{\sum }_{i = 1}^{n} [ \frac{{PV}_{i}}{{( 1 + IRR )}^{i}} - {IF}_{i} ] = 0 \\ {PV}_{i} = {P}_{oil\_i} \times {O}_{P} + {P}_{gas\_i} \times {G}_{P}\end{matrix}$$

wherein P_{oil_i} represents an oil output quantity of an ith year, O_{P} represents an oil price, P_{gas_i} represents a gas output quantity of the ith year, G_{P} represents a natural gas price, PVᵢ represents an output oil and gas value of the ith year, IFᵢ represents an invested capital of the ith year, n represents a production cycle, and IRR represents an ROI.

The embodiments of the present disclosure further provide an apparatus for determining a sweet spot region for shale oil in-situ conversion development, comprising:

a potential index determination module configured to determine an output oil and gas potential index according to a Total Organic Carbon (TOC), a Hydrogen Index (HI) and a shale density;

an effective shale determination module configured to determine a heated shale section according to the output oil and gas potential index and corresponding lower limit value of the oil and gas potential index that is determined according to a well arrangement mode and a shale vitrinite reflectance;

an output quantity determination module configured to determine an output quantity according to a thickness and an area of the heated shale section and data of the output oil and gas potential index;

a Return on Investment (ROI) determination module configured to determine an ROI according to the output quantity and an invested cost; and

a sweet spot region determination module configured to determine a sweet spot region for shale oil in-situ conversion development by using the ROI.

The embodiments of the present disclosure further provide a device for determining a sweet spot region for shale oil in-situ conversion development, comprising a processor and a memory for storing instructions executable by the processor, wherein when being executed by the processor, the instructions implement the steps of:

determining an output oil and gas potential index according to a Total Organic Carbon (TOC), a Hydrogen Index (HI) and a shale density;

determining a heated shale section according to the output oil and gas potential index and corresponding lower limit value of the oil and gas potential index that is determined according to a well arrangement mode and a shale vitrinite reflectance;

determining an output quantity according to a thickness and an area of the heated shale section and data of the output oil and gas potential index;

determining a Return on Investment (ROI) according to the output quantity and an invested cost; and

determining a sweet spot region for shale oil in-situ conversion development by using the ROI.

The embodiments of the present disclosure further provide a system for determining a sweet spot region for shale oil in-situ conversion development, comprising at least one processor and a memory for storing computer executable instructions, wherein when executing the instructions, the processor implements the steps of the method in any one of the above embodiments.

One or more embodiments of present disclosure provide a method, an apparatus and a system for determining a sweet spot region for shale oil in-situ conversion development, which can determine the output oil and gas potential index of the shale section by using the TOC, the HI and the shale density, and further consider the well arrangement mode in the shale oil in-situ conversion development to determine a distribution of the heated shale section, which is favorable for the shale oil development, in the research region. Next, the distribution of the output quantity of the research region can be determined according to the thickness and area of the heated shale section and corresponding output oil and gas potential index, and the distribution of the ROI of the research region can be further determined in combination with the investment cost. By optimally selecting the sweet spot region of shale oil in-situ conversion through the ROI, the accuracy of determination of the shale oil sweet spot region can be greatly improved.

### BRIEF DESCRIPTION OF THE DRAWINGS

In order to more clearly explain the technical solutions in the embodiments of the present disclosure or in the prior art, the drawings to be used in the description of the embodiments or the prior art will be briefly introduced as follows. Obviously, the drawings in the following description merely illustrate some embodiments of the present disclosure, and a person skilled in the art can obtain other drawings from them without paying any creative labor. In which:

FIG. 1 is a schematic flowchart of an embodiment of a method for determining a sweet spot region for shale oil in-situ conversion development provided by the present disclosure;

FIG. 2 is a schematic diagram illustrating a relationship between an effective heated shale thickness and heating time of a single layer linear well pattern model in another embodiment provided by the present disclosure;

FIG. 3 is a schematic diagram illustrating a relationship between upper and lower limit values of a shale thickness, upper limit value of an effective heated shale thickness and the number of well arrangement layers of a heating well in another embodiment provided by the present disclosure;

FIG. 4 is a schematic diagram illustrating a relationship between a lower limit value of an oil and gas potential index and Ro in another embodiment provided by the present disclosure;

FIG. 5 is a schematic diagram illustrating a relationship between ratio of an oil and gas output rate and Ro during shale in-situ conversion in another embodiment provided by the present disclosure;

FIG. 6 illustrates a ratio of an annual oil and gas output quantity to a total oil and gas output quantity in another embodiment provided by the present disclosure;

FIG. 7 is a schematic diagram of a distribution of effective shale thickness of Chang 7 in Ordos basin in another embodiment provided in the present disclosure;

FIG. 8 is a schematic diagram of a distribution of Ro of a heated shale section of Chang 7 in Ordos basin in another embodiment provided in the present disclosure;

FIG. 9 is a schematic diagram of a distribution of oil and gas potential index of a heated shale section of Chang 7 in Ordos basin in another embodiment provided by the present disclosure;

FIG. 10 is a schematic diagram of a distribution of Return on Investment (ROI) of a shale oil in-situ conversion of Chang 7 in Ordos basin in another embodiment provided by the present disclosure;

FIG. 11 is a schematic diagram of a distribution of a sweet spot region for shale oil in-situ conversion of Chang 7 in Ordos basin in another embodiment provided by the present disclosure; and

FIG. 12 is a schematic diagram of module structures of an embodiment of an apparatus for determining a sweet spot region for shale oil in-situ conversion development provided by the present disclosure.

### DETAILED DESCRIPTION OF THE PREFERRED EMBODIMENTS

In order that a person skilled in the art can better understand the technical solutions in the present disclosure, the technical solutions in one or more embodiments of the present disclosure will be described clearly and completely below with reference to the drawings thereof. Obviously, the embodiments described are merely parts, rather than all, of the embodiments of the present disclosure. Based on one or more embodiments in the present disclosure, any other embodiment obtained by a person skilled in the art without paying any creative labor should fall within the protection scope of the present disclosure.

The shale oil is a general designation of generated petroleum hydrocarbons and unconverted organic matters in the organic-rich shale with a burial depth of more than 300 meters and medium and low maturities. The shale with medium and low maturities have extremely low porosity and permeability and poor connectivity, and the flow of fluid therein is difficult.

Specifically, an embodiment of the present disclosure provides a method for determining a sweet spot region for shale oil in-situ conversion development, which determines an output oil and gas potential index of a shale section by using an Total Organic Carbon (TOC), a Hydrogen Index (HI) and a shale density, and reflects oil and gas output potential of a shale interval by using the output oil and gas potential index. The well arrangement mode in shale oil in-situ conversion development is further considered to determine a distribution of a heated shale section, which is favorable for shale oil development, in a research region. Next, a distribution of an output quantity of the research region is determined according to a thickness and an area of the heated shale section and a corresponding output oil and gas potential index, and a distribution of an ROI of the research region is further determined in combination with an invested cost. The sweet spot region for shale oil in-situ conversion are optimally selected through the ROI, thereby greatly improving the accuracy of determination of the shale oil sweet spot region.

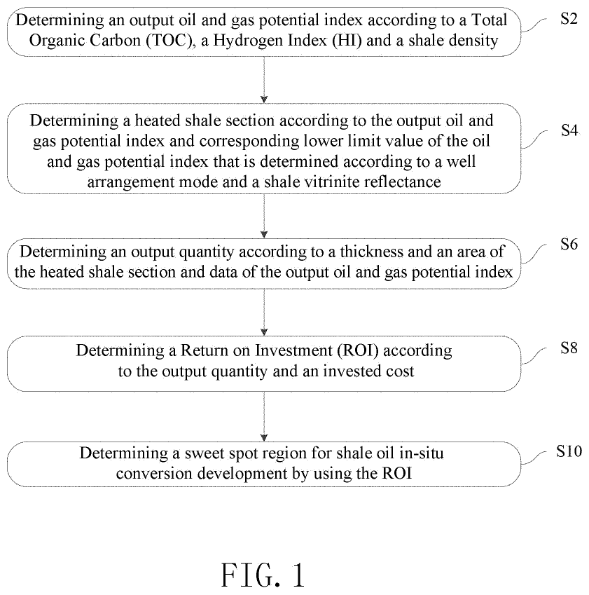

FIG. 1 is a schematic flowchart of an embodiment of a method for determining a sweet spot region for shale oil in-situ conversion development provided by the present disclosure. Although the present disclosure provides the method operation steps or apparatus structures illustrated in the following embodiments or drawings, more operation steps or module units, or less ones after partial combination, may be included in the method or apparatus based on conventional or non-creative labors. In the steps or structures having no necessary causal relationship logically, the execution sequence of these steps or the module structures of the apparatus are not limited to those illustrated in the embodiments of the present disclosure or the drawings. When the method or module structures are applied to the actual apparatus, server or terminal product, they can be executed sequentially or in parallel according to those illustrated in the embodiments or drawings (e.g., under an environment of parallel processors or multithread processing, and even an implementation environment including distributed processing and server cluster).

A specific embodiment is illustrated in FIG. 1. In one embodiment of the method for determining a sweet spot region for shale oil in-situ conversion development provided by the present disclosure, the method may comprise:

S2: determining an output oil and gas potential index according to a Total Organic Carbon (TOC), a Hydrogen Index (HI) and a shale density.

Data of a TOC, an HI, and a shale density ρ of a target stratum in a research region may be measured to determine the output oil and gas potential index according to the measured data.

For example, the vitrinite reflectance Ro at a plurality of measurement points in a longitudinal direction may be measured; if a thickness of a shale of the target stratum in the research region is large, and when a change of Ro of the shale section in the longitudinal direction is greater than 0.1%, it is preferable that the shale section is divided by adopting a change range having an Ro change interval of 0.1%, and an average value of Ro at respective sample points in each of sub-shale sections after division is taken as an Ro value of the sub-shale section.

Next, it is possible to collect logging data of the shale section of the target stratum in the research region and TOC data of a core analysis of corresponding shale section, and calibrate the logging data with the TOC data of the core analysis. The TOC value of the shale section of the target stratum of the evaluated well is acquired through a model Δ log R according to the Ro value of the target stratum in the research region, by using Natural gamma, density, neutron in logging data and acoustic wave logging data, the acquired TOC longitudinal data spacing is the logging measurement point spacing. The logging density data is calibrated with a shale density value of the core analysis of the target stratum in the research region, so as to acquire the shale density value of the shale section of the target stratum in the research region, the acquired shale density longitudinal data spacing is a logging measurement point spacing. Next, a cracked hydrocarbon S₂ of the shale can be acquired according to the core analysis, and the HI of the shale of the target stratum is determined with the following formula: HI=S₂/TOC.

The shale samples of the target stratum are collected at equal spacing for 1 to 10 points per meter, preferably 3 points per meter, so as to collect samples from a shale interval of a coring well of the target stratum in the research region. The samples collected from the shale interval of the same well are pulverized and mixed uniformly, and Ro, TOC, S₂ and ρ are each measured by taking 3 samples mixed uniformly. An average value of Ro of three shale samples is taken as an Ro value of the shale sample of the evaluated well; an average value of HI of three shale samples is taken as an HI value of the shale sample of the evaluated well; and an average value of density of three shale samples is taken as ρ of the shale sample of the evaluated well.

In some embodiments, the vitrinite reflectance Ro of the shale sample of the target stratum in the research region may be measured according to the industrial standard “Measurement Method for Vitrinite Reflectance in Sedimentary Rocks” SY/T 5124-2012, for example. The TOC of the shale sample of the target stratum in the research region is measured according to the national standard “Determination of Total Organic Carbon in Sedimentary Rock” GB/T 19145-2003. S₂ of the shale sample of the target stratum in the research region is measured according to the national standard “Rock Pyrolysis” GB/T 18602-2012, so as to calculate HI. The density ρ of the shale sample of the target stratum in the research region is measured according to the national standard “Method for Measuring Densities of Coal and Rock Mass, Method for Measuring Physical and Mechanical Properties of Coal and Rock, Part 3” GB/T 23561.3-2009.

Next, the distributions of the TOC, HI, and ρ of the research region may be used to further determine the distribution of the output oil and gas potential index of the shale section of the research region. The output oil and gas potential index of the shale section is determined according to the parameters TOC, HI and ρ under different shale vitrinite reflectance Ro, thereby reflecting the potential of the shale interval for in-situ conversion development more accurately and reasonably, which is favorable for determining more accurately the sweet spot region for shale oil development.

In one embodiment of the present disclosure, the output oil and gas potential index of different shale sections may be determined according to the product of TOC, HI and ρ under different shale vitrinite reflectance Ro:

PHI=ρ×TOC×HI (1)

wherein PHI represents an output oil and gas potential index of a shale for in-situ conversion under a certain Ro, TOC represents a total organic carbon of the shale under the Ro, HI represents a hydrogen index of the shale under the Ro, and ρ represents a shale density of the shale under the Ro. Of course, during implementation, the method is not limited to the above calculation way, and a simple modification thereof may also be adopted, for example by adding some constants or power indexes.

S4: determining a heated shale section according to the output oil and gas potential index and corresponding lower limit value of the oil and gas potential index that is determined according to a well arrangement mode and a shale vitrinite reflectance.

The lower limit value of the oil and gas potential index may include a minimum value of the output oil and gas potential index that satisfies a certain ROI. In one embodiment of the present disclosure, the lower limit value of the oil and gas potential index may be predetermined according to the well arrangement mode of the target stratum and the shale vitrinite reflectance. For example, the respective parameters of the exploited region can be counted and analyzed to determine a relationship of change in the lower limit value of the oil and gas potential index relative to the well arrangement mode of the target stratum and the shale vitrinite reflectance, so as to determine lower limit value of the oil and gas potential index of different shale sections in different well arrangement modes.

Next, data of the output oil and gas potential indexes of the longitudinal measurement points of the shale section of the well to be evaluated can be acquired, and an interval where an output oil and gas potential index is greater than or equal to corresponding lower limit value of the oil and gas potential index is determined as an effective shale section.

The heated shale section may include a continuous effective shale section, or include a continuous interval composed of the effective shale sections and the interlayer therebetween. The interlayer is an interval between the effective shale sections, where the oil and gas potential index is smaller than corresponding lower limit value of the oil and gas potential index.

If there are multiple effective shale sections in the target stratum, and the thickness of the interlayer between the effective shale sections is small, the adjacent multiple effective shale sections and the interlayer therebetween may be wholly determined as a heated shale section. If the thickness of the interlayer is large, the effective shale sections above and below the interlayer may be processed separately to determine the heated shale section. In one embodiment of the present disclosure, a continuous shale interval, where a ratio of the thickness of the effective shale to a sum of the thicknesses of the effective shale section and the interlayer is greater than a preset threshold, may be used as a heated shale section, thereby improving the efficiency of subsequent data processing.

In one embodiment of the present disclosure, the effective thermal field distribution of different heating time can be simulated by using software STAR-CMG and designing different well patterns and heating well spacing according to the shale thermal field parameters of the target stratum in the research region, thereby optimizing and determining the well arrangement mode of the target stratum. In one or more embodiments of the present disclosure, the optimized and determined well arrangement mode may include: the heating well pattern perpendicular to the stratum section is arranged by using a linear pattern in single layer or a triangular pattern in two or more layers.

Correspondingly, the reasonable well spacing of the heating well and the effective thermal field thickness distribution may be determined as follows through software simulation STAR-CMG according to the heating time and the thermal field parameters of the target stratum:

It is assumed that the heating time is 4 to 8 years, and preferably 5 years.

The heating well with a linear pattern in single layer has a spacing of 5 to 12 meters, and preferably 8 meters; and the heating well with a triangular pattern in two or more layers has a spacing of 8 to 20 meters, and preferably 12.5 meters.

A ratio of the production wells to the heating wells with a linear pattern in single layer is 1:5 to 1:20, and preferably 1:10; and a ratio of the production wells to the heating wells with a triangular pattern in two or more layers is 1:10 to 1:30, and preferably 1:15.

In one embodiment of the present disclosure, the optimal number of well arrangement layers of the shale section to be evaluated may be optimized and determined according to the lower limit value of the oil and gas potential indexes corresponding to different numbers of well arrangement layers and the upper and lower limit values of the shale thickness. Next, the lower limit value of the oil and gas potential index of the shale section to be evaluated is further determined according to the number of well arrangement layers of the shale section to be evaluated and the shale vitrinite reflectance. The heated shale section of the shale section to be evaluated is determined by using the output oil and gas potential index and the lower limit value of the oil and gas potential index of the shale section to be evaluated, thereby more accurately determining the well arrangement mode and the heated shale section distribution of the shale oil development of the research region.

In some embodiments, it is possible to determine the upper and lower limit values of the shale thickness that can achieve the maximum utilization effect under corresponding number of well arrangement layers of the heating well, according to the heating time, the heating well spacing and the effective thermal field thickness distribution. Under a certain number of well arrangement layers, if the shale thickness is large, the utilization will not be very good, and if the shale thickness is too small, the invested cost may be wasted. Thus, when the shale thickness is between an upper limit value and a lower limit value of a shale thickness under a number of well arrangement layers, the number of well arrangement layers may be adopted as that for the shale thickness, thereby ensuring the maximum utilization effect to maximize the ROI obtained.

In one embodiment of the present disclosure, the upper limit value of the shale thickness corresponding to the number of well arrangement layers n of the heating well may include an upper limit value of an effective heated shale thickness corresponding to the number of well arrangement layers n, and the lower limit value of the shale thickness corresponding to the number of well arrangement layers n of the heating well may include an upper limit value of an effective heated shale thickness corresponding to a number of well arrangement layers n−1, wherein the upper limit value of the effective heated shale thickness may represent a maximum of the effective heated thickness of the whole shale section under the corresponding well arrangement mode and heating time.

In one or more embodiments of the present disclosure, upper limit values of the effective heated shale thickness under different well arrangement modes of the heating well may be determined according to heating time of the preferred heating well, heating well spacing, and effective thermal field thickness distribution by using the following calculation model.

When the well arrangement mode of a triangular pattern of two or more layers is adopted, the upper limit value of the effective heated shale thickness may be calculated by using formula (2):

Heᵤₚ=a₁₁×NL (2)

wherein NL represents the number of well arrangement layers of the heating well, Heᵤₚ represents an upper limit value of the effective heated shale thickness corresponding to NL, and a₁₁ represents a constant, and when the heating well spacing is 12.5 meters, a₁₁ is valued as 10.8.

In particular, when the well arrangement mode of a linear pattern of a single layer is adopted, the upper limit value of the effective heated shale thickness can be calculated by using formula (3):

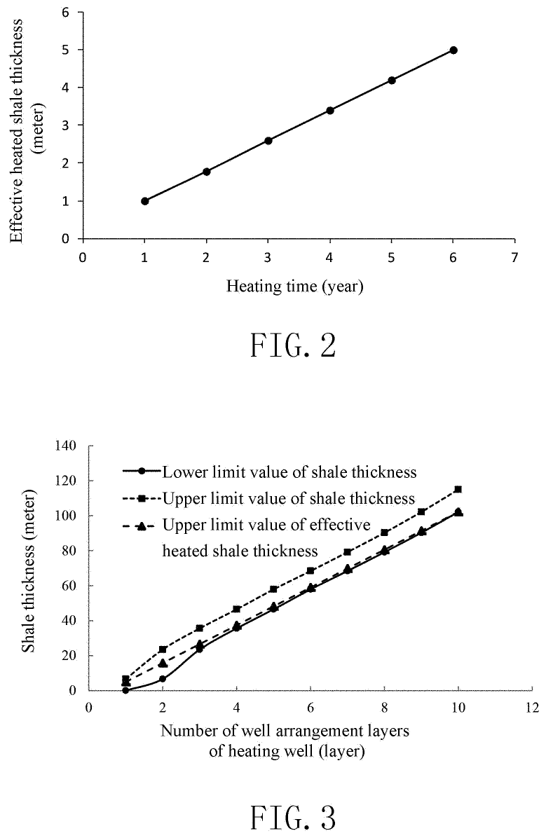

Heᵤₚ=a₂₁×t+a₂₀ (3)

wherein He^{up} represents an upper limit value of the effective heated shale thickness, t represents heating time, and a₂₁ and a₂₀ represent constants, which are valued as 0.800171 and 0.19067, respectively. As illustrated in FIG. 2, which is a schematic diagram illustrating a relationship between an effective heated shale thickness and heating time of a single layer linear well pattern model.

In some embodiments of the present disclosure, the comprehensive cost and ROI of the unit oil and gas output of the shale oil in-situ conversion may be further considered to determine the upper and lower limit values of the shale thickness under different number of well arrangement layers of the heating well. In one or more embodiments of the present disclosure, the upper and lower limit values of the shale thickness may be determined according to the following calculation model of the shale thickness:

$${H}_{up} or {H}_{down} = \{ \begin{matrix}{a}_{33} \times {NL}^{3} + {a}_{32} \times {NL}^{2} + {a}_{31} \times NL + {a}_{30} & NL \leq 4 \\ {b}_{31} \times NL + {b}_{30} & NL > 4\end{matrix} \qquad ( 4 )$$

wherein NL represents the number of well arrangement layers of the heating well, Hᵤₚ represents an upper limit value of the shale thickness corresponding to NL, H_{down} represents a lower limit value of the shale thickness corresponding to NL, and a₃₃, a₃₂, a₃₁, a₃₀, b₃₁ and b₃₀ represent constants. Table 1 shows the values of a₃₃, a₃₂, a₃₁, a₃₀, b₃₁ and b₃₀₀f a certain research region, wherein H_{down} of the NL layers is equal to Hᵤₚ of the NL-1 layers. As illustrated in FIG. 3, which is a schematic diagram illustrating a relationship between upper and lower limit values of a shale thickness, upper limit value of an effective heated shale thickness and the number of well arrangement layers of a heating well.

When the upper limit value of the effective shale thickness of the target stratum in the research region (the total effective shale thickness in a shale section to be arranged) is greater than or equal to H_{down} of the NL layers and less than Hᵤₚ of the NL layers, the ROI is the maximum by arranging the well in NL layers, and thus the heating well in NL layers may be adopted.

TABLE 1
Empirical Parameters in Shale Thickness Calculation Model

| | Parameter | | | | | |
|---|---|---|---|---|---|---|
| | a₃₃ | a₃₂ | a₃₁ | a₃₀ | b₃₁ | b₃₀ |
| Upper limit value | 0.5833 | −5.85 | 30.2667 | −18.3 | 11.2857 | 0.9286 |
| of shale thickness | | | | | | |
| Lower limit value | −2.4667 | 19.85 | −35.5833 | 18.2 | 11.025 | −8.5893 |
| of shale thickness | | | | | | |

In some embodiments of the present disclosure, the upper limit value of the effective heated shale thickness can be taken as the heated shale thickness under the corresponding well arrangement mode according to the upper limit values of the effective heated shale thicknesses under different well arrangement modes. According to the heated shale thickness, the data of the output oil and gas potential index satisfying a preset minimum ROI is determined by using relevant parameters, and taken as the lower limit value of the oil and gas potential index under corresponding well arrangement mode and Ro. Next, the above data can be taken as sample data to analyze the relationship of change in the lower limit value of the oil and gas potential index relative to the number of well arrangement layers and the shale vitrinite reflectance.

Further, the above sample data may be used to construct a calculation model of the lower limit value of the oil and gas potential index under different well arrangement mode of the heating well, and the lower limit value of the oil and gas potential index of the target stratum under different well arrangement modes can be determined according to the calculation model of the lower limit value of the oil and gas potential index. In one embodiment of the present disclosure, the calculation model of the lower limit value of the oil and gas potential index may include:

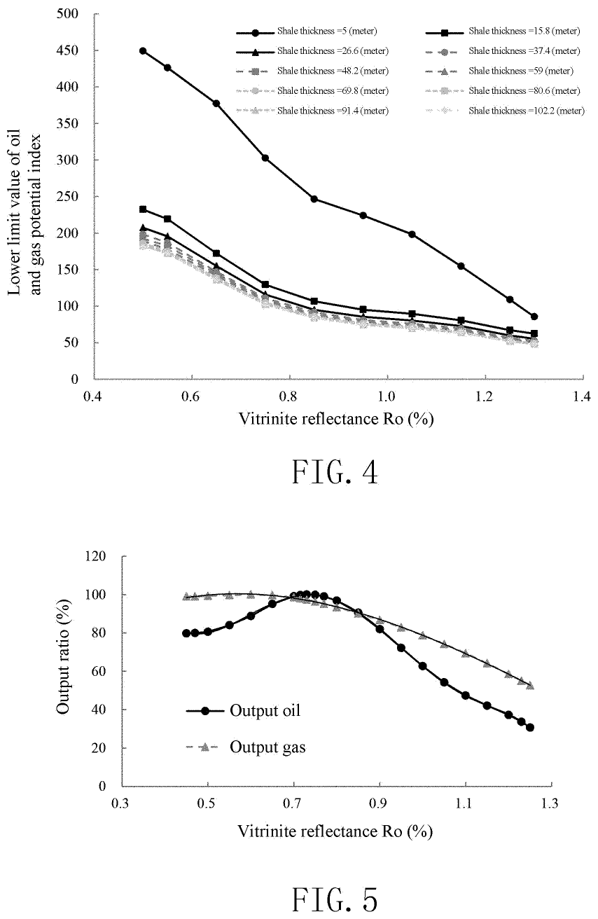

PHI_{cutof}=100×(a₈₅×Ro⁵+a₈₄×Ro⁴+a₈₃×Ro³+a₈₂×Ro²+a₈₁×Ro+a₈₀) (5)

wherein PHI_{cutof} represents an lower limit value of the output oil and gas potential index, Ro represents a vitrinite reflectance, and a₈₀, a₈₁, a₈₂, a₈₃, a₈₄ and a₈₅ represents constants. Table 2 shows the values of a₈₀ to a₈₅ of a research region. FIG. 4 is a schematic diagram illustrating a relationship between a lower limit value of an oil and gas potential index and Ro. As can be seen from FIG. 4, as the shale thickness decreases, the minimum output oil and gas potential index (i.e., the lower limit value of the oil and gas potential index) required to reach a certain ROI increases. Therefore, a larger exploitation benefit cannot be well achieved by determining the sweet spot region only according to some geological parameters.

TABLE 2
Empirical Parameters in the Calculation Model of the Lower
limit value of the oil and gas potential index

| Number Of | | | | | | |
|---|---|---|---|---|---|---|
| well | | | | | | |
| arrangement | Parameter | | | | | |
| layer | a₈₅ | a₈₄ | a₈₃ | a₈₂ | a₈₁ | a₈₀ |
| 1 layer | 106.88 | −481.65 | 839.09 | −701.88 | 275.64 | −36.136 |
| 2 layers | 67.68 | −301.31 | 517.47 | −423.79 | 161.36 | −20.52 |
| 3 layers | 61.88 | −276.39 | 476.28 | −391.55 | 149.88 | −19.29 |
| 4 layers | 53.15 | −237.95 | 410.58 | −337.35 | 128.51 | −16.17 |
| 5 layers | 57.55 | −257.72 | 445.46 | −367.53 | 141.33 | −18.34 |
| 6 layers | 51.89 | −232.66 | 402.28 | −331.53 | 126.91 | −16.16 |
| 7 layers | 51.07 | −228.39 | 393.81 | −323.65 | 123.52 | −15.65 |
| 8 layers | 49.95 | −223.63 | 386.00 | −317.47 | 121.18 | −15.33 |
| 9 layers | 49.56 | −222.14 | 383.78 | −315.85 | 120.61 | −15.27 |
| 10 layers | 52.78 | −236.58 | 409.14 | −337.61 | 129.72 | −16.75 |

In some embodiments of the present disclosure, based on the determination methods of the lower limit value of the oil and gas potential index and the upper and lower limit values of the shale thickness provided in the above embodiments, the number of well arrangement layers of the shale section to be evaluated can be optimized and determined and the heated shale section distribution can be determined as follows.

Firstly, the lower limit value of the oil and gas potential index corresponding to the well arrangement mode under the maximum number m of well arrangement layers of the heating well (preferably, 10 layers of the heating well) can be taken as a criterion. PHI_{cutof}^{m} of the shale sections of the evaluated well is determined according to the Ro thereof, and the data of the output oil and gas potential indexes of the longitudinal measurement points of the shale sections of the evaluated well are calculated to take a shale section where an output oil and gas potential index is greater than or equal to PHI_{cutof}^{m} as the effective shale section. In addition, the distribution of the heated shale section of the shale sections of the evaluated well is further determined according to the solution in the above embodiment. In order to facilitate the subsequent description, the heated shale section determined according to PHI_{cutof}^{m} may be called as an initial heated shale section in this embodiment.

When the thickness of the initial heated shale is greater than or equal to the lower limit value H_{down} of the shale thickness corresponding to the heating well pattern of n layers and less than Hᵤₚ, the lower limit value PHI_{cutof}ⁿ of the oil and gas potential index corresponding to the well arrangement mode of the heating well pattern of n layers is taken as a criterion to redetermine the distribution of the heated shale section of the shale sections of the evaluated well according to the data of the output oil and gas potential indexes of the longitudinal measurement points of the shale sections of the evaluated well.

When the thickness of the heated shale redetermined according to PHI_{cutof}ⁿ is greater than or equal to H_{down} of the heating well pattern of n layers and less than Hᵤₚ of the heating well pattern of n layers, the thickness of the heated shale redetermined according to PHI_{cutof}ⁿ is taken as the thickness of the shale for the final evaluation.

When the recalculated thickness of the heated shale is less than H_{down} of the heating well pattern of n layers and greater than H_{down} of the heating well pattern of n−1 layers, the lower limit value PHI_{cutof}ⁿ⁻¹ of the oil and gas potential index corresponding to the well arrangement mode of the heating well pattern of n−1 layers is taken as a criterion to redetermine the distribution of the heated shale section of the shale sections of the evaluated well.

The rest can be done in the same manner until the thickness of the heated shale section satisfies the parameter range value of corresponding well arrangement mode, so as to optimize and determine the number of well arrangement layers of the shale sections of the evaluated well, and the distribution of the heated shale section according to the above solution. Next, the heating well spacing may be optimized and determined according to the number of well arrangement layers and the thickness of the heated shale. By adopting the solution of the above embodiment, it is possible to more accurately determine the distribution of the heated shale section and the well arrangement mode corresponding to each heated shale section.

S6: determining an output quantity according to a thickness and an area of the heated shale section and data of the output oil and gas potential index.

It is possible to acquire the distribution of the heated shale section determined in the above step, and then analyze the data of thickness and area of each heated shale section, and acquire the data of TOC, HI and shale density ρ in each heated shale section. For example, it is possible to count the data of TOC, HI and shale density ρ of the measurement points of each heated shale section, and calculate the output oil and gas potential index of each measurement point. The average value of the output oil and gas potential index of each measurement point is taken as the output oil and gas potential index of corresponding heated shale section. Next, it is possible to determine the output quantity of corresponding heated shale section according to the thickness, area and output oil and gas potential index of the heated shale section, wherein the output quantity may include an oil output quantity and a gas output quantity.

In other embodiments of the present disclosure, it is possible to acquire the data of thickness and area of each effective shale section in the heated shale section, and the output oil and gas potential index of each effective shale section. Next, the output quantity of corresponding heated shale section is determined according to the thickness, area, and output oil and gas potential index of each effective shale section, thereby eliminating the influence of the interlayer in the heated shale section on the calculation result.

In one embodiment of the present disclosure, an output rate may be determined according to the output oil and gas potential index and a ratio of output rate, and the output quantity may be calculated according to the output rate of the heated shale section as well as the thickness, area, and shale density thereof.

The output rate may include an oil output rate and a gas output rate, which may include output oil quality, gas volume in unit shale quality, respectively. The ratio of output rate may include a ratio of oil output rate and a ratio of gas output rate in shale oil in-situ conversion, may include percentages of the oil output quantity and the gas output quantity of the shale in the maximum oil output quantity and the maximum gas output quantity, respectively, under different Ro during shale oil in-situ conversion.

In one or more embodiments of the present disclosure, the ratio of oil output rate or the ratio of gas output rate of the target stratum may be determined according to a pre-built percentage calculation model:

wherein the calculation model for the ratio of oil output rate may be represented as:

PRo=100×(a₄₆×Ro⁶+a₄₅×Ro⁵+a₄₄×Ro⁴+a₄₃×Ro³+a₄₂×Ro²+a₄₁×Ro+a₄₀) (5)

the calculation model for the ratio of gas output rate may be represented as:

PRg=100×(a₅₃×Ro³+a₅₂×Ro²+a₅₁×Ro+a₅₀) (6)

wherein PRO represents a ratio of oil output rate, PRg represents a ratio of gas output rate, Ro represents a shale vitrinite reflectance, and a₄₀, a₄₁, a₄₂, a₄₃, a₄₄, a₄₅, a₄₆, a₅₀, a₅₁, a₅₂ and a₅₃ represent empirical parameters. Table 3 shows the values of a₄₆ to a₄₀ and a₅₃ to a₅₀ of a certain research region, and FIG. 5 is a schematic diagram illustrating a relationship between the ratio of oil output rate and the ratio of gas output rate and Ro during shale in-situ conversion.

TABLE 3
Empirical Parameters of Models for Ratio of oil output rate and Ratio
of gas output rate during Shale Oil in-situ Conversion

| | Parameter | | | | | | |
|---|---|---|---|---|---|---|---|
| | a₄₆ | a₄₅ | a₄₄ | a₄₃ | a₄₂ | a₄₁ | a₄₀ |
| Ratio | −58.544 | 250.958 | −405.158 | 296.376 | −87.225 | 0.529 | 3.690 |
| of oil | | | | | | | |
| output | | | | | | | |
| rate | | | | | | | |

Parameter

| | a₅₃ | a₅₂ | a₅₁ | a₅₀ |
|---|---|---|---|---|
| Ratio of gas | 0.539 | −2.308 | 2.101 | 0.457 |
| output rate | | | | |

In one embodiment of the present disclosure, the oil and gas output rates of the shale in-situ conversion of the target stratum in the research region may be acquired through formula (7) according to the oil output rate, the gas output rate, the empirical data of TOC, Ro and HI, the ratio of oil output rate and the ratio of gas output rate of the in-situ conversion of existing shale under the geological condition similar to that of the target stratum in the research region, as well as the data of TOC, Ro and HI of the shale of the target stratum in the research region:

$${Qf}_{1} = {Qf}_{2} \times \frac{{ρ}_{1} \times {PR}_{1} \times {TOC}_{1} \times {HI}_{1}}{{ρ}_{2} \times {PR}_{2} \times {TOC}_{2} \times {HI}_{2}} \qquad ( 7 )$$

wherein Qf₁ represents an oil output rate and a gas output rate of a shale of the target stratum in the research region, Qf₂ represents an oil output rate and a gas output rate of an in-situ conversion of existing shale oil under a geological condition similar to that of the target stratum in the research region, PR₁ represents a ratio of oil output rate and a ratio of gas output rate of the target stratum in the research region under Ro, PR₂ represents a ratio of oil output rate and a ratio of gas output rate of a thermal simulation of existing shale under Ro and a geological condition similar to that of the target stratum in the research region, TOC₁ represents a TOC value of the target stratum in the research region, TOC₂ represents a TOC value of an oil and gas output rate sample of an in-situ conversion of existing shale oil under a geological condition similar to that of the target stratum in the research region, HI₁ represents an HI value of the target stratum in the research region, HI₂ represents an HI value of an oil and gas output rate sample of an in-situ conversion of existing shale oil under a geological condition similar to that of the target stratum in the research region, ρ₁ represents a shale density value of the target stratum in the research region, and ρ₂ represents a shale density value of an oil and gas output rate sample of an in-situ conversion of existing shale oil under a geological condition similar to that of the target stratum in the research region.

Correspondingly, ρ×TOC×HI in formula (7) represents an oil and gas potential index, and formula (7) may be represented as:

$${Qf}_{1} = {Qf}_{2} \times \frac{{PR}_{1} \times {PHI}_{1}}{{PR}_{2} \times {PHI}_{2}} \qquad ( 8 )$$

wherein PHI₁ represents an output oil and gas potential index of a shale of a target stratum in a research region, and PHI₂ represents an output oil and gas potential index of an existing shale under a geological condition similar to that of the target stratum in the research region.

Table 4 shows the oil and gas outputs and relevant parameters in an in-situ conversion of a hermetic coring well of existing Chang 7 shale in Ordos Basin calculated through the solution of the above embodiment.

TABLE 4
Oil and Gas Outputs and Relevant Parameters in In-Situ Conversion of
Hermetic Coring Well of Chang 7 Shale in Ordos Basin

| TOC | | | | Oil output rate | Gas output rate |
|---|---|---|---|---|---|
| (%) | HI (mg/g) | ρ (g/cm³) | Ro (%) | (kg/t · rk) | (m³/t · rk) |
| 23.7 | 353 | 2.01 | 0.81 | 55.27 | 26.05 |

Next, it is possible to calculate the oil output rate and gas output rate of the heated shale section in the above manner, and then determine the output quantity of the heated shale section according to the thickness, the horizontal distribution area and the density average value of the heated shale section. In some embodiments, it is possible to calculate the output quantity according to formulas (9) and (10):

Pₒᵢₗ=10⁻⁷×Qfₒᵢₗ×He×A×ρ_{rock} (9)

P_{gas}=10⁻⁴×Qf_{gas}×He×A×ρ_{rock} (9)

wherein Pₒᵢₗ represents a total oil output quantity in a production cycle, P_{gas} represents a total gas output quantity in the production cycle, Qfₒᵢₗ represents an oil output rate of a heated shale section, Qf_{gas} represents a gas output rate of the heated shale section, He represents a thickness of an effective shale in the heated shale section, A represents an area of the effective shale section in the heated shale section, and ρ_{rock} represents a density average value of the effective shale section in the heated shale section.

S8: determining a Return on Investment (ROI) according to the output quantity and an invested cost.

It is possible to acquire the invested cost of oil and gas output per unit in the shale oil in-situ conversion by considering fees such as the fixed investment, the operating cost, the tax, the reclamation fees, etc. of the oil and gas output per unit in the shale oil in-situ conversion. The ROI is determined according to the output quantity and the invested cost.

In one embodiment of the present disclosure, the ROI may be determined according to the following ROI calculation model:

$${\sum }_{i = 1}^{n} [ \frac{{PV}_{i}}{{( 1 + IRR )}^{i}} - {IF}_{i} ] = 0 \qquad ( 11 )$$

wherein PVᵢ represents an output oil and gas value of an ith year, IFᵢ represents an invested capital of the ith year, n represents a production cycle, and IRR represents an ROI.

wherein the output oil and gas value may be determined according to an oil price when a shale oil in-situ conversion development is performed, and under a certain oil price:

PVᵢ=P_{oil_i}×O_{P}+P_{gas_i}×G_{P} (12)

wherein P_{oil_i} represents an oil output quantity of the ith year, O_{P} represents an oil price, P_{gas_i} represents a natural gas output quantity of the ith year, and G_{P} represents a natural gas price.

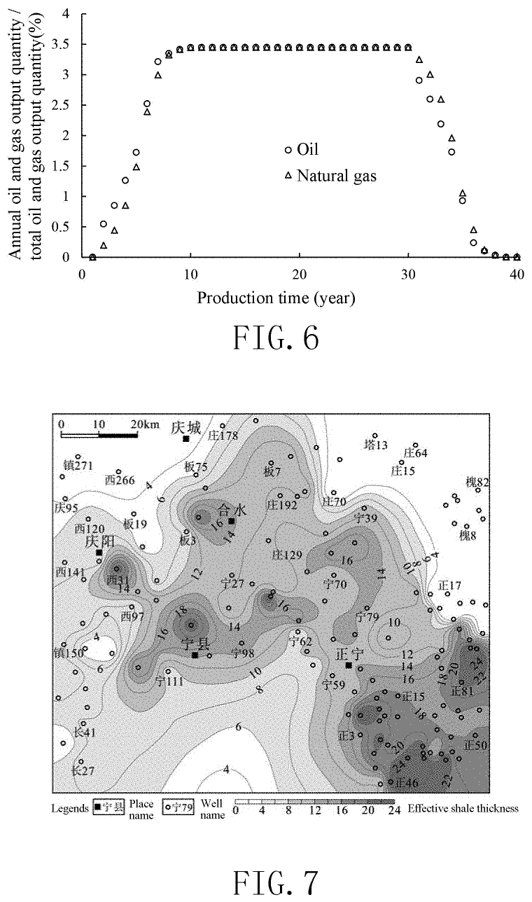

Under different production cycles or development modes, a ratio of an annual oil and gas output quantity to a total oil and gas output quantity is varied. For example, during a production cycle of 40 years, a ratio of an annual oil and gas output quantity to a total oil and gas output quantity is calculated by using the ratio illustrated in FIG. 6.

P_{oil_i}=10⁻²×Pₒᵢₗ×R_{oil_i} (13)

P_{gas_i}=10⁻²×P_{gas}×R_{gas_i} (14)

wherein R_{oil_i} represents a ratio of an oil output quantity in the ith year to a total oil output quantity within a production cycle, and R_{gas_i} represents a ratio of a gas output quantity in the ith year to a total gas output quantity within the production cycle.

IFᵢ=Capexᵢ+Opexᵢ+Taxᵢ+Dctᵢ (15)

wherein Capexᵢ represents a fixed investment in the ith year, Opexᵢ represents an operating cost of the ith year, Taxᵢ represents a tax of the ith year, and Dctᵢ represents a waste investment.

S10: determining a sweet spot region for shale oil in-situ conversion development by using the ROI.

It is possible to acquire the ROI of the well point in the research region, acquire planar distribution of the ROI of the target stratum in the research region by interpolation method, and analyze the planar distribution of the ROI to determine the sweet spot region for the shale oil in-situ conversion development. In some embodiments, a region, where an ROI is greater than a lower limit value of the ROI and a continuous distribution area is greater than a lower limit value of the area, may be determined as a sweet spot region. Preferably, the lower limit value of the area may be 10 km², and the lower limit value of ROI may be 8%.

In some embodiments of the present disclosure, it is possible to filter the regions that satisfy a preset condition, and then determine the sweet spot region for the shale oil in-situ conversion development according to the ROI. The preset condition may include: Ro of the shale of the target stratum ranges from 0.2% to 1.1%, and the kerogen is of type I to II; there is no active water in the region, and the water content of shale is less than 5%, preferably less than 2%; the heated shale section has a sealing stratum with good sealing property, wherein the sealing layer refers to a mudstone or gypsum rock salt stratum that is in direct contact with the heated section of the shale at a top or bottom of the heated shale section, and the good sealing property means that a thickness of the sealing stratum is greater than 2 meters, preferably 5 meters. No fracture or fault grows in the heated shale section and the sealing stratum in the region, and the burial depth is less than 4000 meters, preferably 3000 meters, such that the sweet spot region can be determined more accurately.

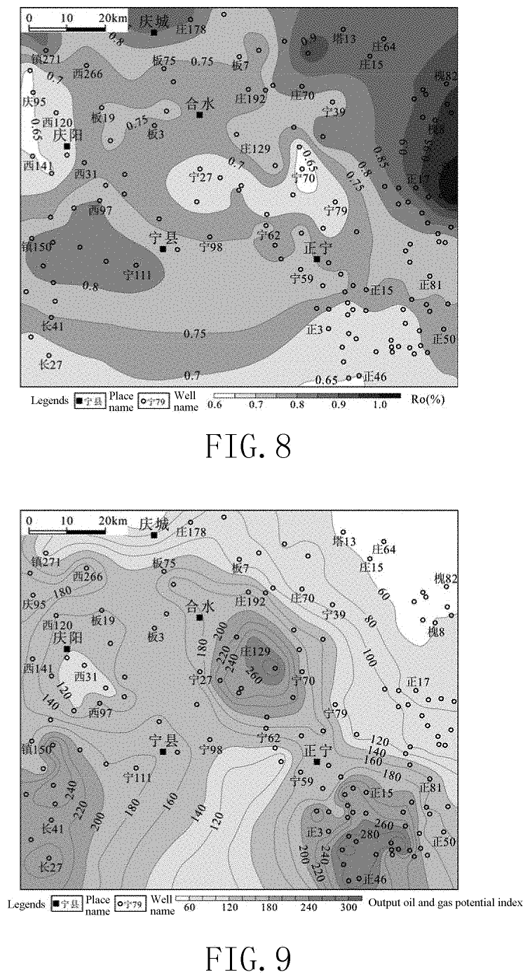

According to the above method provided by the embodiment of the present disclosure, the target stratum in the research region of Chang 7 in Ordos basin is analyzed to determine the sweet spot region. The planar distribution of the thickness of the determined effective shale section (the effective shale section in the heated shale section) is illustrated in FIG. 7, the planar distribution of Ro of the heated shale section is illustrated in FIG. 8, and the planar distribution of the output oil and gas potential index of the heated shale section is illustrated in FIG. 9.

The power generation mode of self-built power plant is adopted for the heating of the in-situ conversion, the natural gas output quantity of the sweet spot region of the target stratum in the research region is larger than the quantity of the natural gas used for power generation, and the remaining natural gas after the consumption for power generation is not taken into account for the value. The fixed investment expenses on drilling and heater, etc., the operating costs and the taxes are calculated based on the actual cost of the current oilfield, and the reclamation fee is calculated as 4% of the total investment fee; the oil price is 60 $/barrel, the crude oil production capacity is calculated as 2.5 million tons/year, and the production time is calculated as 39 years. Through the above solution, the planar distribution of ROI of the heated shale section of Chang 7 is determined, as illustrated in FIG. 10.

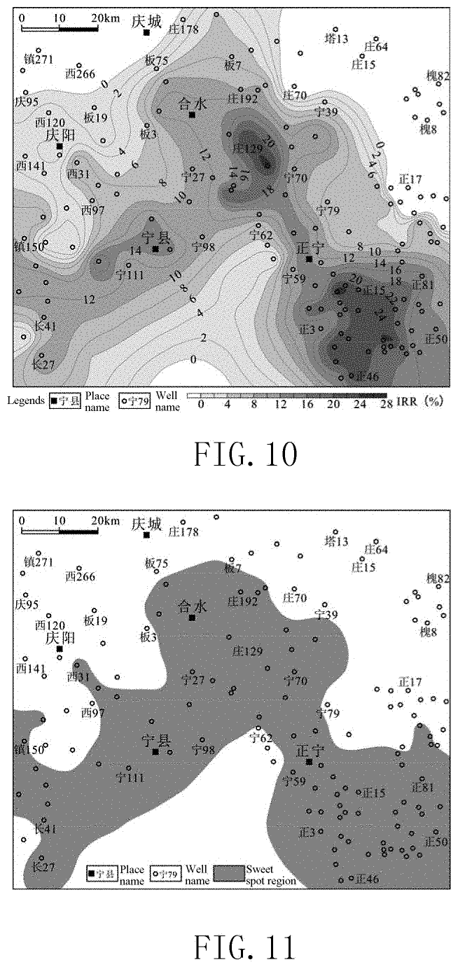

According to the lower limit criterion that the ROI is greater than or equal to 8%, the sweet spot region for the shale oil in-situ conversion of a heated shale section of a target stratum in a research region in Chang 7 in Ordos basin is determined as illustrated in FIG. 11. Within the evaluated range of 23748 km², the sweet spot region has an area of 9770 km², about 12 billion tons of economically recoverable oil resources, about 5.2 trillion cubic meters of economically recoverable natural gas resources, and about 15 billion tons of oil equivalent.

According to the above solution provided by the embodiment of the present disclosure, the sweet spot region for the shale oil in-situ conversion is optimally selected by using the ROI evaluation by combining the well arrangement mode, the thickness of the heated shale and the output oil and gas potential index in the shale oil in-situ conversion development with the economic evaluation, thereby solving the problem that the sweet spot region is not accurately selected by solely considering the geological factor. In addition, the above solution proposes the lower limit value of the output oil and gas potential index of the sweet spot region under different Ro and the well arrangement modes of different heating well patterns, fully considers the shale oil output potential, and provides guarantee for improving the accuracy of evaluation and optimal selection of the sweet spot region. Further, the above solution proposes the condition and criterion for the optimal selection of the sweet spot region for shale oil in-situ conversion, thereby providing achievable approaches and methods for the evaluation and optimal selection of the sweet spot region. Therefore, by adopting the solution of the embodiment of the present disclosure, the benefit of the shale oil in-situ conversion development can be greatly improved.

The embodiments of the present disclosure are all described in a progressive manner, and the same or similar portions of the embodiments can refer to each other. Each embodiment lays an emphasis on its distinctions from other embodiments. Specifically, references may be made to the description of the previous embodiments of related processing, which will be omitted herein.

The particular embodiments of the present disclosure have been described above. Other embodiments fall within the scope of the appended claims. In some cases, the actions or steps recited in the claims may be performed in a different order than in the embodiments and still achieve the desired results. In addition, the processes depicted in the drawings do not necessarily require the illustrated particular order or consecutive order to achieve the desired results. In some embodiments, multitask processing and parallel processing are also possible or favorable.

One or more embodiments of present disclosure provide a method for determining a sweet spot region for shale oil in-situ conversion development, which can determine the output oil and gas potential index of the shale section by using the TOC, the HI and the shale density, and further consider the well arrangement mode in the shale oil in-situ conversion development to determine a distribution of the heated shale section in the research region favorable for the shale oil development. Next, the distribution of the output quantity of the research region can be determined according to the thickness and area of the heated shale section and corresponding output oil and gas potential index, and the distribution of the ROI of the research region can be further determined in combination with the investment cost. By optimally selecting the sweet spot region of shale oil in-situ conversion through the ROI, the accuracy of determination of the shale oil sweet spot region can be greatly improved.

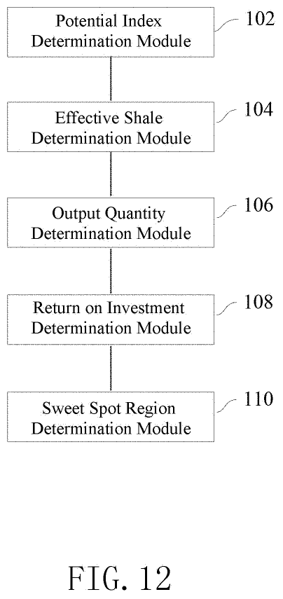

Based on the above method for determining a sweet spot region for shale oil in-situ conversion development, one or more embodiments of present disclosure further provide an apparatus for determining a sweet spot region for shale oil in-situ conversion development, which may include means using systems, software (applications), modules, components, servers, etc. involved in the method described in the embodiments of the present disclosure and combining necessary implementation hardware. Based on the same innovative concept, the apparatus(es) in one or more embodiments provided by the present disclosure will be described in the following embodiments. Since the implementation solution of the apparatus to solve the problem is similar to that of the method, the implementation of the specific apparatus in the embodiments of the present disclosure may refer to that of the aforementioned method, which will not be repeated. As used below, the term “unit” or “module” may be a combination of software and/or hardware that implements a predetermined function. Although the apparatus described in the following embodiments is preferably implemented in software, implementations of hardware, or a combination of software and hardware, are also possible and contemplatable. Specifically, FIG. 12 is a schematic structure diagram of modules of an embodiment of an apparatus for determining a sweet spot region for shale oil in-situ conversion development provided by the present disclosure. As illustrated in FIG. 12, the apparatus may comprise:

a potential index determination module 102, which may be configured to determine an output oil and gas potential index according to a Total Organic Carbon (TOC), a Hydrogen Index (HI) and a shale density;

an effective shale determination module 104, which may be configured to determine a heated shale section according to the output oil and gas potential index and corresponding lower limit value of the oil and gas potential index that is determined according to a well arrangement mode and a shale vitrinite reflectance;

an output quantity determination module 106, which may be configured to determine an output quantity according to a thickness and an area of the heated shale section and data of the output oil and gas potential index;

a Return on Investment (ROI) determination module 108, which may be configured to determine an ROI according to the output quantity and an invested cost; and

a sweet spot region determination module 110, which may be configured to determine a sweet spot region for shale oil in-situ conversion development by using the ROI. It should be noted that the apparatus described above may also include other embodiments according to the description of the method embodiments. The specific implementations may refer to the description of related method embodiments and will not be repeated herein.

One or more embodiments of present disclosure provide an apparatus for determining a sweet spot region for shale oil in-situ conversion development, which can determine the output oil and gas potential index of the shale section by using the TOC, the HI and the shale density, and further consider the well arrangement mode in the shale oil in-situ conversion development to determine a distribution of the heated shale section in the research region favorable for the shale oil development. Next, the distribution of the output quantity of the research region can be determined according to the thickness and area of the heated shale section and corresponding output oil and gas potential index, and the distribution of the ROI of the research region can be further determined in combination with the investment cost. By optimally selecting the sweet spot region of shale oil in-situ conversion through the ROI, the accuracy of determination of the sweet spot region of the shale oil can be greatly improved.

The method or apparatus described in the above embodiments provided in the present disclosure may realize a service logic by a computer program and record it in a storage medium that is readable and executable by a computer to achieve the effects of the solutions described in the embodiments of the present disclosure. Thus, the present disclosure further provides a device for determining a sweet spot region for shale oil in-situ conversion development, comprising a processor and a memory for storing instructions executable by the processor, wherein the instructions implement the following steps when being executed by the processor:

determining an output oil and gas potential index according to a TOC, an HI and a shale density;

determining a heated shale section according to the output oil and gas potential index and corresponding lower limit value of the oil and gas potential index that is determined according to a well arrangement mode and a shale vitrinite reflectance;

determining an output quantity according to a thickness and an area of the heated shale section and data of the output oil and gas potential index data;

determining a Return on Investment (ROI) according to the output quantity and an invested cost;

determining a sweet spot region for shale oil in-situ conversion development by using the ROI.

The storage medium may include a physical device for storing information that is usually digitized and then stored in a medium using electronic, magnetic or optical manner, etc. The storage medium may further include a device that stores information by means of electric energy, such as RAM and ROM; a device that stores information by means of magnetic energy, such as hard disk, floppy disk, magnetic tape, magnetic core memory, magnetic bubble memory and U disk; and a device that stores information optically, such as CD or DVD. Of course, there may be other forms of readable storage mediums, such as a quantum memory, a graphene memory, etc.

It should be noted that the above processing device according to the description of method embodiments may further include other embodiments. The specific implementations may refer to the description of related method embodiments, and will not be repeated herein.

The apparatus for determining a sweet spot region for shale oil in-situ conversion development described in the above embodiment can determine the output oil and gas potential index of the shale section by using the TOC, the HI and the shale density, and further consider the well arrangement mode in the shale oil in-situ conversion development to determine a distribution of the heated shale section, which is favorable for the shale oil development, in the research region. Next, the distribution of the output quantity of the research region can be determined according to the thickness and area of the heated shale section and corresponding output oil and gas potential index, and the distribution of the ROI of the research region can be further determined in combination with the investment cost. By optimally selecting the sweet spot region of shale oil in-situ conversion through the ROI, the accuracy of determination of the shale oil sweet spot region can be greatly improved.

The present disclosure further provides a system for determining a sweet spot region for shale oil in-situ conversion development, which may be a separate system for determining a sweet spot region, and may also be applied in a shale oil in-suit development system. For example, it may be software (application), actual operation device, logic gate circuit device, quantum computer, etc., and may be a terminal device combining necessary implementation hardware. The system for determining a sweet spot region comprises at least one processor and a memory for storing computer executable instructions, wherein when executing the instructions, the processor implements the steps of the method in any one of the above embodiments.

It should be noted that the system described above may also comprise other embodiments according to the description of method or apparatus embodiments. The specific implementation may refer to the description of related method embodiments and will not be repeated herein.

The system for determining a sweet spot region for shale oil in-situ conversion development described in the above embodiment can determine the output oil and gas potential index of the shale section by using the TOC, the HI and the shale density, and further consider the well arrangement mode in the shale oil in-situ conversion development to determine a distribution of the heated shale section, which is favorable for the shale oil development, in the research region. Next, the distribution of the output quantity of the research region can be determined according to the thickness and area of the heated shale section and corresponding output oil and gas potential index, and the distribution of the ROI of the research region can be further determined in combination with the investment cost. By optimally selecting the sweet spot region of shale oil in-situ conversion through the ROI, the accuracy of determination of the shale oil sweet spot region can be greatly improved.

It should be noted that the apparatus or system described above in the present disclosure may also include other embodiments according to the description of the method embodiments. The specific implementations may refer to the description of related method embodiments and will not be repeated herein. The embodiments of the present disclosure are all described in a progressive manner, and the same or similar portions of the embodiments can refer to each other. Each embodiment lays an emphasis on its distinctions from other embodiments. In particular, the embodiments such as hardware+program and storage medium+program are simply described since they are substantially similar to the method embodiment, and please refer to the description of the method embodiment for the relevant portions.

Although the operations and the data description such as the acquisition, definition, interaction, calculation, judgment, etc. of the output oil and gas potential index, the heated shale section, etc. are mentioned in the embodiments of the present disclosure, the embodiments of the present disclosure are not limited to those that must meet the standard data model/template or the situations described in the embodiments of the present disclosure. Some industrial standards or self-defined embodiments or those slightly modified based on the implementations described in the above embodiments may achieve the same, equivalent or similar, or modification-predictable implementation effects of the above embodiments. The embodiments obtained by applying the amended or modified data acquisition, storage, judgment, processing methods may still fall within the scope of optional embodiments of the present disclosure.

The particular embodiments of the present disclosure have been described above. Other embodiments fall within the scope of the appended claims. In some cases, the actions or steps recited in the claims may be performed in a different order than in the embodiments and still achieve the desired results. In addition, the processes depicted in the drawings do not necessarily require the illustrated particular order or consecutive order to achieve the desired results. In some embodiments, multitask processing and parallel processing are also possible or favorable.

Any system, apparatus, module or unit set forth in the embodiments specifically may be implemented by a computer chip or an entity, or by a product having a certain function. A typical implementation device is a computer. Specifically, the computer may be, for example, a personal computer, a laptop computer, a vehicle-mounted man-machine interaction device, a tablet computer, or a combination of any of these devices.

For the convenience of description, the above apparatus is described as various modules in terms of functions. Of course, when implementing one or more embodiments of the present disclosure, the functions of various modules may be realized in the same one or more software and/or hardware, and a module that realizes the same function may also be implemented by a combination of a plurality of sub-modules or sub-units, etc. The apparatus embodiment described above is only illustrative. For example, the division of the units is only a logical function division. In actual implementation, there may be other division methods. For example, a plurality of units or components may be combined or integrated into another system, or some features may be ignored or not implemented. On the other hand, the coupling or direct coupling or communication connection between each other illustrated or discussed may be indirect coupling or communication connection through some interfaces, devices or units, and may be in electrical, mechanical or other forms.

A person skilled in the art also know that it is entirely possible to perform logic programming on the method steps, such that the controller realizes the same function in a form of logic gate, switch, application-specific integrated circuit, programmable logic controller, embedded microcontroller, etc., excepting implementing the controller in the form of pure computer readable program codes. Thus, such a controller may be considered as a hardware component, and means comprised therein for implementing various functions may also be considered as structures within the hardware component. Or even, the means for realizing various functions may be regarded as both software modules for realizing a method and structures within the hardware component.

The present disclosure is described with reference to a flow diagram and/or a block diagram of the method, apparatus (system) and computer program product according to the embodiments of the present disclosure. It should be appreciated that each flow and/or block in the flow diagram and/or the block diagram and a combination of flows and/or blocks in the flow diagram and/or the block diagram can be realized by computer program instructions. Those computer program instructions can be provided to a general computer, a dedicated computer, an embedded processor or a processor of other programmable data processing device to produce a machine, so that the instructions executed by the processor of the computer or other programmable data processing device produce a means for realizing specified functions in one or more flows in the flow diagram and/or one or more blocks in the block diagram.

These computer program instructions may also be stored in a computer readable memory capable of guiding the computer or other programmable data processing devices to work in a particular manner, so that the instructions stored in the computer readable memory can produce manufacture articles including an instructing device which realizes function(s) specified in one or more flows in the flow diagram and/or one or more blocks in the block diagram.

These computer program instructions may also be loaded onto the computer or other programmable data processing devices, so that a series of operation steps are performed on the computer or other programmable data processing devices to produce a processing realized by the computer, thus the instructions executed on the computer or other programmable devices provide step(s) for realizing function(s) specified in one or more flows in the flow diagram and/or one or more blocks in the block diagram.

In a typical configuration, the computing device comprises one or more processors (CPUs), an input/output interface, a network interface and a memory.

Further to be noted, the term “comprise”, “include” or any other variant intends to cover the non-exclusive inclusions, so that a process, a method, a commodity or a device comprising a series of elements comprise not only those elements, but also other elements not explicitly listed, or further comprise inherent elements of such process, method, commodity or device. In a case where there is no further limitation, the elements defined by a sentence “comprising a . . . ” do not exclude other identical elements existing in the process, method, commodity or device comprising the elements.

A person skilled in the art should appreciate that one or more embodiments of the present disclosure can be provided as a method, a system or a computer program product. Therefore, the one or more embodiments of the present disclosure can take the form of a full hardware embodiment, a full software embodiment, or an embodiment combining software and hardware. Moreover, the one or more embodiments of the present disclosure can take the form of a computer program product implemented on one or more computer usable storage mediums (including, but not limited to, a magnetic disc memory, CD-ROM, optical storage, etc.) containing therein computer usable program codes.

One or more embodiments of the present disclosure may be described in the general context of computer executable instructions executed by the computer, e.g., the program module. In general, the program module includes routine, program, object, component, data structure, etc. executing a particular task or realizing a particular abstract data type. One or more embodiments of the present disclosure may also be put into practice in the distributed computing environments where tasks are executed by remote processing devices connected through a communication network. In the distributed computing environments, the program modules may be located in the local and remote computer storage medium including the storage device.

The embodiments of the present disclosure are all described in a progressive manner, and the same or similar portions of the embodiments can refer to each other. Each embodiment lays an emphasis on its distinctions from other embodiments. In particular, the system embodiment is simply described since it is substantially similar to the method embodiment, and please refer to the description of the method embodiment for the relevant portions. In the description of the present disclosure, the description of reference terms “one embodiment”, “some embodiments”, “examples”, “specific examples” or “some examples”, and the like mean that the specific features, structures, materials, or characteristics described in conjunction with the embodiment(s) or example(s) are included in at least one embodiment or example of the present disclosure. In the present disclosure, the schematic expressions of the above terms do not necessarily aim at the same embodiment or example. Moreover, the specific features, structures, materials, or characteristics described may be combined in any one or more embodiments or examples in a suitable manner. In addition, a person skilled in the art may combine different embodiments or examples described in the present disclosure and features thereof if there is no contradiction

Those described above are just embodiments of the present disclosure, rather than limitations to the present disclosure. For a person skilled in the art, the present disclosure is intended to cover various amendments or variations. Any amendment, equivalent substitution, improvement, etc. made under the spirit and principle of the present disclosure should fall within the scope of the claims of the present disclosure.

What is claimed is:

1. A method for determining a sweet spot region for shale oil in-situ conversion development, comprising:
determining an output oil and gas potential index according to a Total Organic Carbon (TOC), a Hydrogen Index (HI) and a shale density;
determining a heated shale section according to the output oil and gas potential index and corresponding lower limit value of an oil and gas potential index that is determined according to a well arrangement mode and a shale vitrinite reflectance;
determining an output quantity according to a thickness and an area of the heated shale section and data of the output oil and gas potential index;
determining a Return on Investment (ROI) according to the output quantity and an invested cost; and
determining a sweet spot region for shale oil in-situ conversion development by using the ROI.

2. The method for determining a sweet spot region for shale oil in-situ conversion development according to claim 1, wherein the determining a heated shale section according to the output oil and gas potential index and corresponding lower limit value of an oil and gas potential index comprises:
determining an interval, where the output oil and gas potential index is greater than or equal to corresponding lower limit value of the oil and gas potential index, as an effective shale section; and
determining a continuous shale interval, where a ratio of a thickness of the effective shale to thicknesses of the effective shale section and an interlayer between the effective shale sections is greater than a preset threshold, as the heated shale section.

3. The method for determining a sweet spot region for shale oil in-situ conversion development according to claim 1, wherein the determining a heated shale section according to the output oil and gas potential index and corresponding lower limit value of an oil and gas potential index comprises:
determining the number of well arrangement layers of a shale section to be evaluated, according to the lower limit values of the oil and gas potential indexes corresponding to different numbers of well arrangement layers and upper and lower limit values of a shale thickness;
determining the lower limit value of the oil and gas potential index of the shale section to be evaluated, according to the number of well arrangement layers of the shale section to be evaluated and a shale vitrinite reflectance; and
determining the heated shale section of the shale section to be evaluated, according to the output oil and gas potential index and the lower limit value of the oil and gas potential index of the shale section to be evaluated.

4. The method for determining a sweet spot region for shale oil in-situ conversion development according to claim 1, wherein the determining an output quantity according to a thickness and an area of the heated shale section and data of the output oil and gas potential index comprises:
determining an output rate of the heated shale section according to data of the output oil and gas potential index and ratio of the output rate of the heated shale section; and
calculating the output quantity according to the output rate of the heated shale section as well as the thickness, the area, and the shale density thereof.

5. The method for determining a sweet spot region for shale oil in-situ conversion development according to claim 1, wherein the well arrangement mode includes: a heating well pattern perpendicular to a stratum section is arranged by using a linear pattern in single layer or a triangular pattern in two or more layers.

6. The method for determining a sweet spot region for shale oil in-situ conversion development according to claim 3, wherein the determining the lower limit value of the oil and gas potential index according to the number of well arrangement layers and a shale vitrinite reflectance comprises:
determining the lower limit value of the oil and gas potential index of a target stratum according to a pre-built calculation model for the lower limit value of the oil and gas potential index, the calculation model includes:
PHI_{cutof}=100×(a₈₅×Ro⁵+a₈₄×Ro⁴+a₈₃×Ro³+a₈₂×Ro²+a₈₁×Ro+a₈₀)
wherein PHI_{cutof} represents a lower limit value of an oil and gas potential index, Ro represents a vitrinite reflectance, and a₈₀, a₈₁, a₈₂, a₈₃, a₈₄ and a₈₅ represent constants and are determined according to the number of well arrangement layers.

7. The method for determining a sweet spot region for shale oil in-situ conversion development according to claim 3, wherein upper and lower limit values of the shale thickness are calculated according to a shale thickness calculation model as follows:
$${H}_{up} or {H}_{down} = \{ \begin{matrix}{a}_{33} \times {NL}^{3} + {a}_{32} \times {NL}^{2} + {a}_{31} \times NL + {a}_{30} & NL \leq 4 \\ {b}_{31} \times NL + {b}_{30} & NL > 4\end{matrix}$$
wherein NL represents the number of well arrangement layers of a heating well, Hᵤₚ represents an upper limit value of a shale thickness of corresponding to NL, H_{down} represents a lower limit value of the shale thickness of the corresponding to NL, and a₃₃, a₃₂, a₃₁, a₃₀, b₃₁ and b₃₀ represent constants.

8. The method for determining a sweet spot region for shale oil in-situ conversion development according to claim 4, wherein a ratio of the output rate of the heated shale section is calculated according to a pre-built percentage calculation model as follows:
$$\{ \begin{matrix}PRo = 100 \times ( {a}_{46} \times {Ro}^{6} + {a}_{45} \times {Ro}^{5} + {a}_{44} \times {Ro}^{4} + {a}_{43} \times {Ro}^{3} + {a}_{42} \times {Ro}^{2} + {a}_{41} \times Ro + {a}_{40} ) \\ PRg = 100 \times ( {a}_{53} \times {Ro}^{3} + {a}_{52} \times {Ro}^{2} + {a}_{51} \times Ro + {a}_{50} )\end{matrix}$$
wherein PRO represents a ratio of oil output rate, PRg represents a ratio of gas output rate, Ro represents a shale vitrinite reflectance, and a₄₀, a₄₁, a₄₂, a₄₃, a₄₄, a₄₅, a₄₆, a₅₀, a₅₁, a₅₂ and a₅₃ represent constants.

9. The method for determining a sweet spot region for shale oil in-situ conversion development according to claim 1, wherein the determining a Return on Investment (ROI) according to the output quantity and an invested cost comprises:
$$\{ \begin{matrix}{\sum }_{i = 1}^{n} [ \frac{{PV}_{i}}{{( 1 + IRR )}^{i}} - {IF}_{i} ] = 0 \\ {PV}_{i} = {P}_{oil\_i} \times {O}_{P} + {P}_{gas\_i} \times {G}_{P}\end{matrix}$$
wherein P_{oil_i} represents an oil output quantity of an ith year, O_{P} represents an oil price, P_{gas_i} represents a gas output quantity of the ith year, G_{P} represents a natural gas price, PVᵢ represents an output oil and gas value of an ith year, IFᵢ represents an invested capital of the ith year, n represents a production cycle, and IRR represents an Return on Investment.

10. An apparatus for determining a sweet spot region for shale oil in-situ conversion development, comprising:
a potential index determination module configured to determine an output oil and gas potential index according to a Total Organic Carbon (TOC), a Hydrogen Index (HI) and a shale density;
an effective shale determination module configured to determine a heated shale section according to the output oil and gas potential index and corresponding lower limit value of the oil and gas potential index that is determined according to a well arrangement mode and a shale vitrinite reflectance;
an output quantity determination module configured to determine an output quantity according to a thickness and an area of the heated shale section and data of the output oil and gas potential index;
a Return on Investment (ROI) determination module configured to determine an ROI according to the output quantity and an invested cost; and
a sweet spot region determination module configured to determine a sweet spot region for shale oil in-situ conversion development by using the ROI.

11. A device for determining a sweet spot region for shale oil in-situ conversion development, comprising a processor and a memory for storing instructions executable by the processor, wherein when being executed by the processor, the instructions implement the steps of:
determining an output oil and gas potential index according to a Total Organic Carbon (TOC), a Hydrogen Index (HI) and a shale density;
determining a heated shale section according to the output oil and gas potential index and corresponding lower limit value of an oil and gas potential index that is determined according to a well arrangement mode and a shale vitrinite reflectance;
determining an output quantity according to a thickness and an area of the heated shale section and data of the output oil and gas potential index;
determining a Return on Investment (ROI) according to the output quantity and an invested cost; and
determining a sweet spot region for shale oil in-situ conversion development by using the ROI.

12. A system for determining a sweet spot region for shale oil in-situ conversion development, comprising at least one processor and a memory for storing computer executable instructions, wherein when executing the instructions, the processor implements the steps of the method according to claim 1.
